(19) United States
(12) Patent Application Publication
(10) Pub. No.: US 2022/0409016 A1
(43) Pub. Date: Dec. 29, 2022

(54) ENDOSCOPE SYSTEM, CONTROL APPARATUS, EXTERNAL APPARATUS, COMBINATION OF CONTROL APPARATUS AND EXTERNAL APPARATUS, AND METHOD OF CHECKING ENDOSCOPE SYSTEM CONFIGURATION

(71) Applicant: OLYMPUS CORPORATION, Tokyo (JP)

(72) Inventor: Tadataka MAYUZUMI, Tokyo (JP)

(73) Assignee: OLYMPUS CORPORATION, Tokyo (JP)

(21) Appl. No.: 17/939,188

(22) Filed: Sep. 7, 2022

Related U.S. Application Data
(63) Continuation of application No. PCT/JP2021/004758, filed on Feb. 9, 2021.

(30) Foreign Application Priority Data
Mar. 9, 2020 (JP) 2020-040224

(51) Int. Cl. A61B 1/00; H04N 5/232

(52) U.S. Cl. CPC A61B 1/00055; H04N 5/232941; H04N 5/232945; H04N 5/23206; A61B 1/00006; A61B 1/0005; A61B 1/00011; H04N 2005/2255

(57) ABSTRACT

An endoscope system includes an endoscope, a control apparatus to which the endoscope is connected, at least one accessory device used in combination with the endoscope and the control apparatus, and data tags respectively provided in the endoscope and the accessory device. The control apparatus reads first data of the respective data tags, compares second data respectively registered about the endoscope and the accessory device and the first data, and performs warning when the first data and the second data do not coincide.

### CROSS REFERENCE TO RELATED APPLICATION

This application is a continuation application of PCT/JP2021/004758 filed on Feb. 9, 2021 and claims benefit of Japanese Application No. 2020-040224 filed in Japan on Mar. 9, 2020, the entire contents of which are incorporated herein by this reference.

### BACKGROUND OF THE INVENTION

### 1. Field of the Invention

The present invention relates to an endoscope system, a control apparatus, an external apparatus, a combination of the control apparatus and the external apparatus, and a method of checking an endoscope system configuration.

### 2. Description of the Related Art

An endoscope system is widely used in the medical field and the industrial field. The endoscope system is used in combination with a plurality of accessory devices. An endoscope and various accessory devices used in a test or the like are selected by a user (for example, a doctor) who performs the test or the like and set in a test room or the like before the test or the like is started.

Among the accessory devices used in combination with the endoscope, there is an accessory device that does not conform to an endoscope other than a specific endoscope. There are a large number of accessory devices used in the endoscope system. Accordingly, it is likely that an accessory device not conforming to an endoscope to be used is erroneously set. For example, Japanese Patent Application Laid-Open Publication No. 2005-58450 proposes an endoscope management system that determines whether an accessory device used in combination with an endoscope conforms to the endoscope.

### SUMMARY OF THE INVENTION

An endoscope system according to an aspect of the present invention includes: an endoscope; a control apparatus to which the endoscope is connected; at least one accessory device used in combination with the endoscope and the control apparatus; and data tags respectively provided in the endoscope and the accessory device. The control apparatus reads first data of the respective data tags, compares second data respectively registered about the endoscope and the accessory device and the first data, and performs warning when the first data and the second data do not coincide.

A control apparatus according to an aspect of the present invention is a control apparatus to which an endoscope is connected and that is used in combination with an accessory device, the control apparatus reading first data of data tags respectively provided in the endoscope and the accessory device, comparing second data respectively registered about the endoscope and the accessory device and the first data, and performing warning when the first data and the second data do not coincide.

A control apparatus according to an aspect of the present invention is a control apparatus to which an endoscope is connected and that is used in combination with an accessory device and connected to an external apparatus via a network, the control apparatus reading first data of data tags respectively provided in the endoscope and the accessory device and, based on a result of comparison by the external apparatus between second data respectively registered about the endoscope and the accessory device and the first data, performing warning when the first data and the second data do not coincide.

A combination of a control apparatus and an external apparatus according to an aspect of the present invention is a combination of a control apparatus and an external apparatus to which an endoscope is connected and that are used in combination with an accessory device, the control apparatus reading first data of data tags respectively provided in the endoscope and the accessory device, the external apparatus comparing second data respectively registered about the endoscope and the accessory device and the first data, and the control apparatus performing warning when the first data and the second data do not coincide.

An external apparatus according to an aspect of the present invention is an external apparatus to which a control apparatus to which an endoscope is connected and that is used in combination with an accessory device is connected via a network, the external apparatus comparing first data of data tags respectively provided in the endoscope and the accessory device and second data respectively registered about the endoscope and the accessory device and transmitting a comparison result to the control apparatus.

A method of checking an endoscope system configuration according to an aspect of the present invention is a method of checking a system configuration of an endoscope system including an endoscope, a control apparatus to which the endoscope is connected, and an accessory device used in combination with the endoscope and the control apparatus, the control apparatus reading first data of data tags respectively provided in the endoscope and the accessory device, comparing second data respectively registered about the endoscope and the accessory device and the first data, and performing warning when the first data and the second data do not coincide.

### BRIEF DESCRIPTION OF THE DRAWINGS

FIG. 1 is a configuration diagram showing a configuration of an endoscope system according to an embodiment;

FIG. 2 is a diagram for explaining a configuration relating to a system configuration check function of the endoscope system according to the embodiment;

FIG. 3 is a diagram showing an example of a device-to-be-used registration screen for registering accessory devices to be used according to the embodiment;

FIG. 4 is a diagram showing a display example of a pulldown menu display section at the time when a type of an endoscope is set according to the embodiment;

FIG. 5 is a diagram showing a display example of the pulldown menu display section at the time when a type of a treatment instrument is set according to the embodiment;

FIG. 6 is a diagram showing an example of a state in which accessory devices are selected on the device-to-be-used registration screen according to the embodiment;

FIG. 7 is a flowchart showing an example of a flow of registration processing for an accessory device by a device-to-be-used registration program according to the embodiment;

FIG. 8 is a flowchart showing an example of a flow of processing of a system configuration check program according to the embodiment;

FIG. 9 is a diagram showing a display example of a monitor, which is an example of a fail-safe operation, according to the embodiment;

FIG. 10 is a diagram showing a display example of a message in a message display section according to the embodiment;

FIG. 11 is a diagram showing a display example of the monitor that is an example of the fail-safe operation, in which the message display section is arranged on an endoscopic image display region, according to the embodiment; and

FIG. 12 is a diagram showing a display example of the monitor at the time when all of accessory devices registered by a user are collected according to the embodiment.

### DETAILED DESCRIPTION OF THE PREFERRED EMBODIMENTS

An embodiment of the present invention is explained below with reference to the drawings.

### (Configuration)

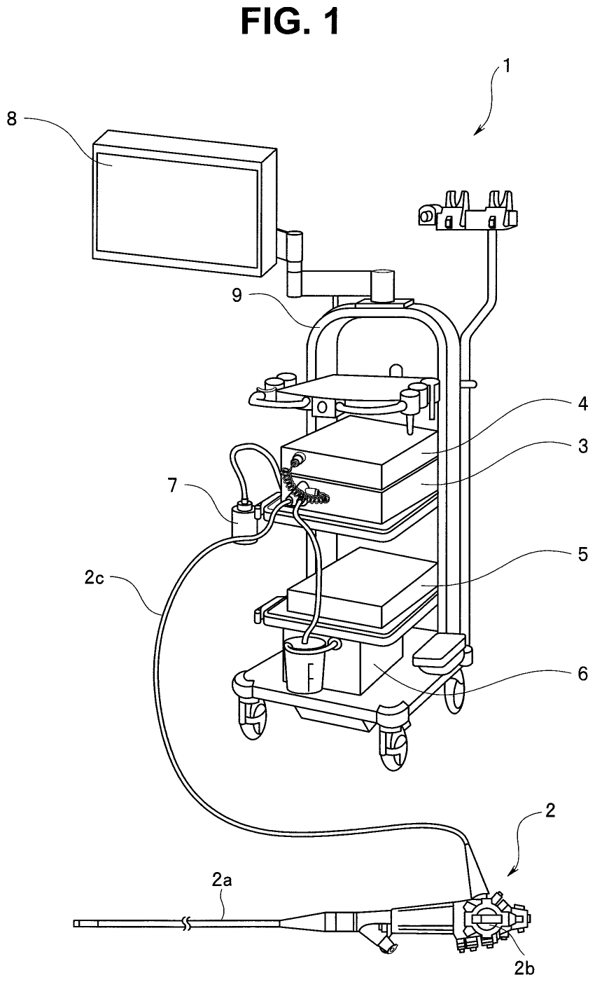

FIG. 1 is a configuration diagram showing a configuration of an endoscope system according to the present embodiment. An endoscope system 1 includes an endoscope 2, a light source apparatus 3 to which the endoscope 2 is connected, a video processor 4, an insertion shape detection apparatus 5 (hereinafter, UPD 5), a suction pump 6, a liquid feeding tank 7, and a monitor 8. As shown in FIG. 1, the light source apparatus 3, the video processor 4, the UPD 5, the suction pump 6, the liquid feeding tank 7, and the monitor 8 are mounted on or fixed to a cart 9.

The endoscope system 1 is disposed in, for example, a test room where a test and treatment for a subject are performed. Besides in the test room, a treatment instrument necessary for the test and the treatment and accessory devices (not shown) such as a mouthpiece are also disposed near the endoscope system 1. As explained above, the endoscope system 1 includes the endoscope 2, the video processor 4 functioning as a control apparatus to which the endoscope 2 is connected, and the accessory devices (the light source apparatus 3, the liquid feeding tank 7, the mouthpiece, the treatment instrument, and the like) used in combination with the endoscope 2 and the video processor 4.

For example, prior to an endoscopic examination, a user determines the endoscope 2, the video processor 4, and the accessory devices (the UPD 5, the suction pump 6, the liquid feeding tank 7, the monitor 8, the treatment instrument, and the like) necessary for the endoscopic examination. The accessory devices also include, for example, a mouthpiece having a desired size and a desired shape, a valve for an operation button set in an operation section 2b, a liquid feeding tank having a desired capacity, and a suction pump having a desired suction force. The user determines an accessory device matching a purpose of a test and having a desired feeling of use and a desired operation feeling.

The endoscope 2, the video processor 4 to which the endoscope 2 is connected, and an accessory device used in combination with the endoscope 2 and the video processor 4, which are determined by the user, are set in the test room by the user, a nurse, or the like.

Note that as explained below, the user registers, in the video processor 4, in advance before a treatment start, data (second data) of the endoscope 2, the video processor 4, and the accessory device determined as being used in the test.

The endoscope 2 includes an elongated insertion section 2a, an operation section 2b provided at a proximal end of the insertion section 2a, and a universal cable 2c extended from the operation section 2b. A connector provided at a distal end of the universal cable 2c is connected to the light source apparatus 3. A cable extending from the connector provided at the distal end of the universal cable 2c is connected to the video processor 4. In other words, the endoscope 2 is connected to the video processor 4, which is the control apparatus.

The light source apparatus 3 incorporates a light source such as a light emitting diode or a halogen lamp and emits light to a proximal end face of a light guide (not shown) inserted through an inside of the endoscope 2 connected to the light source apparatus 3. The light of the light source apparatus 3 made incident on the light guide is emitted from a distal end face of the light guide as illumination light.

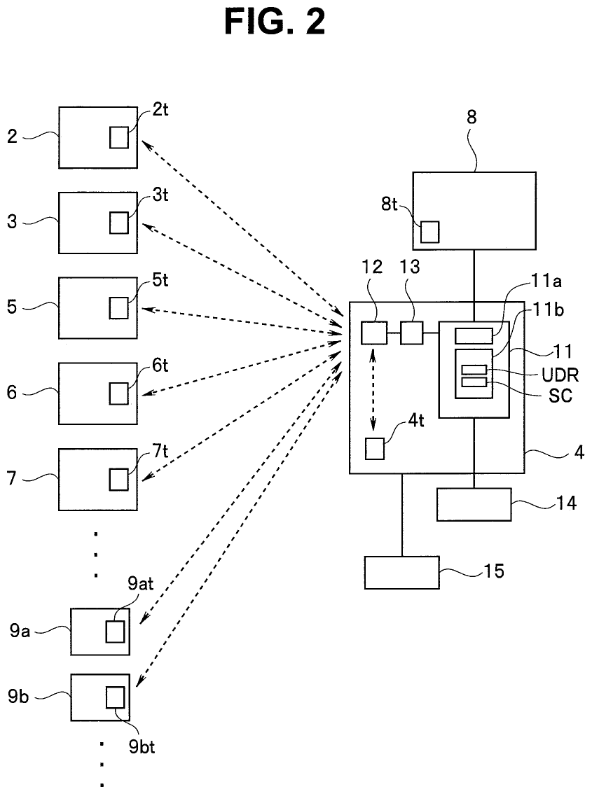

The video processor 4 is the control apparatus used together with the endoscope 2. The video processor 4 receives an image pickup signal from the endoscope 2 via a signal line inserted through the universal cable 2c and subjects the received image pickup signal to image processing to generate an endoscopic image. A video signal of the generated endoscopic image is outputted from the video processor 4 to the monitor 8. The endoscopic image is displayed on a display screen of the monitor 8. An input apparatus 14 (FIG. 2) including a keyboard and a mouse is connected to the video processor 4. As explained below, the video processor 4 includes a system configuration check program SC (FIG. 2) explained below.

The UPD 5 detects a shape of the insertion section 2a and displays the detected shape of the insertion section 2a on a not-shown display apparatus. A surgeon can recognize an insertion state of the insertion section 2a in the subject by viewing the insertion shape of the insertion section 2a displayed on the display apparatus.

The suction pump 6 sucks liquid in a body cavity when a test or the like is performed. The endoscope 2 has a suction function. The suction pump 6 is driven when the suction function is executed.

The liquid feeding tank 7 stores saline ejected into a body when the test or the like is performed.

The monitor 8 includes a display screen for displaying an image. The monitor 8 receives a video signal from the video processor 4 and displays an image including an endoscopic image on the display screen.

Further, as explained above, in the test or the like, besides, accessory devices such as a mouthpiece and a valve set in the operation section 2b of the endoscope 2 are also used.

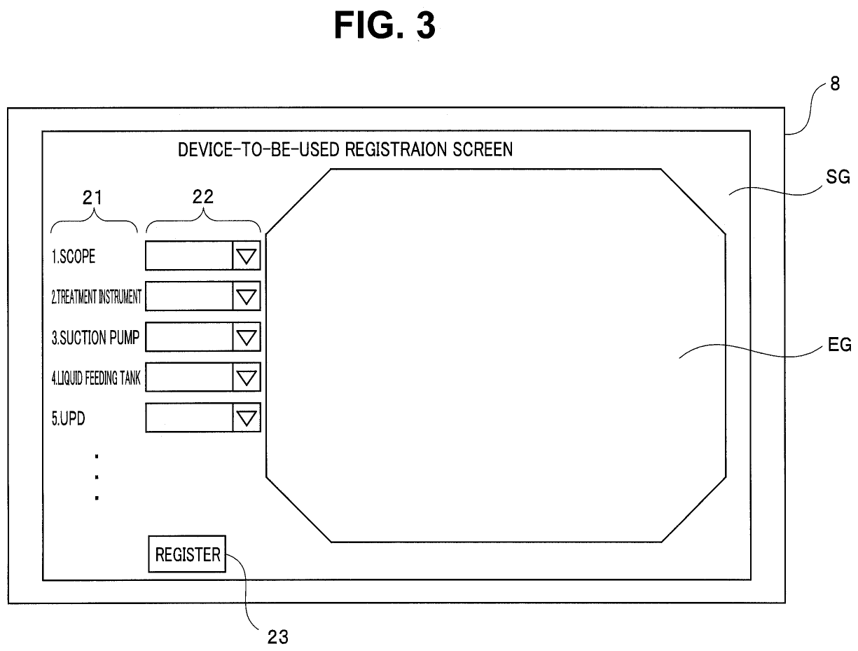

The user registers, in advance, data of the determined endoscope 2, and accessory devices such as the light source apparatus 3 and the UPD 5 in the video processor 4. As explained below, the user can select and register, using a device-to-be-used registration screen SG, a type of the endoscope 2, a type of the video processor 4, possibility of use of the UPD 5, a type of the monitor 8, and the like. The user can also register, using the device-to-be-used registration screen SG (FIG. 3), a type of the treatment instrument, a type of the valve set in the operation section 2b, a type of the mouthpiece, a type of the suction pump, and the like.

The registered respective accessory devices are necessary for the test and are devices intended to use by the user. By using an endoscope system including a plurality of devices intended by the user, the user can perform the test while operating the respective devices with a desired feeling of use and a desired operation feeling.

FIG. 2 is a diagram for explaining a configuration relating to the system configuration check function of the endoscope system 1. A system configuration check is performed by a control unit 11 provided in the video processor 4. It is confirmed by the system configuration check whether all accessory devices used in combination with the endoscope 2 and the video processor 4 are collected when the video processor 4 is energized, that is, turned on.

RFID (radio frequency identification) tags capable of performing wireless communications are attached to the respective accessory devices used for the endoscopic examination. As shown in FIG. 2, RFID tags 2t, 3t, 4t, 5t, 6t, 7t, and 8t are respectively attached to the endoscope 2 and the accessory devices (the light source apparatus 3, the video processor 4, the UPD 5, the suction pump 6, the liquid feeding tank 7, and the monitor 8). RFID tags 9at and 9bt are also respectively embedded in accessory devices 9a and 9b such as a guide tube for insertion guide for the insertion section 2a of the endoscope 2, a mouthpiece attached to a mouth of the subject, a valve for operation button of the endoscope, and a treatment instrument. In other words, the RFID tags are respectively provided in the endoscope 2 and the plurality of accessory devices. In the respective RFID tags, data (first data) of names and types of the accessory devices is recorded. Note that IC tags or circuit boards capable of performing wireless communication may be used instead of the RFID tags. Information media that can read data of an incorporated memory using an electromagnetic wave like the RFID tags, the IC tags, the circuit boards, and the like capable of performing wireless communication are herein defined as “data tags”. In the following description, the “data tags” conform to this definition.

The video processor 4 includes an antenna 12 for performing wireless communication with the respective data tags and a reception circuit 13 connected to the antenna 12. The reception circuit 13 is controlled by the control unit 11. The control unit 11 can acquire signals received from the respective data tags by the reception circuit 13.

Communication between the antenna 12 and the respective data tags is according to, for example, a communication scheme in which a passive microwave is used. An electromagnetic wave reaching distance of the communication scheme in which the passive microwave is used is a maximum of approximately 2 meters.

Note that for the communication between the antenna 12 and the respective data tags, a communication scheme other than the communication scheme in which the passive microwave is used, for example, a communication scheme in which a radio wave in a passive UHF band (for example, a 900 MHz band) having an electromagnetic wave reaching distance of a maximum of 5 meters is used or a communication scheme in which an active radio wave (up to 2.45 GHz band) having an electromagnetic wave reaching distance of a maximum of several ten meters is used may be used.

The control unit 11 receives an image pickup signal from the endoscope 2, generates an endoscopic image, and outputs a video signal for an image for display including the endoscopic image to the monitor 8. The control unit 11 performs control of various devices such as the endoscope 2 included in the endoscope system 1 for various functions for the endoscopic examination. Further, the control unit 11 executes system configuration check processing explained below. The control unit 11 includes a processor 11a and a storage device 11b.

The processor 11a includes a central processing unit (CPU), a ROM, and a RAM. The storage device 11b includes a rewritable nonvolatile memory such as a flash memory or a hard disk device. The CPU of the processor 11a reads out and executes various software programs stored in the ROM and the storage device 11b, whereby various functions are realized. A program for performing a system configuration check is stored in the storage device 11b and is executed when a predetermined command is inputted. More specifically, a device-to-be-used registration program UDR and a system configuration check program SC are stored in the storage device 11b.

Note that a part of the processor 11a may be configured by an integrated circuit such as an FPGA (field programmable gate array), an electronic circuit, and the like.

As explained above, the user registers accessory devices used in the test or the like in the video processor 4 before the test. FIG. 3 is a diagram showing an example of a device-to-be-used registration screen for registering accessory devices to be used. When the user operates, for example, the keyboard of the input apparatus 14 and inputs a predetermined command, the device-to-be-used registration program UDR is executed and the device-to-be-used registration screen shown in FIG. 3 is displayed on a screen of the monitor 8.

The device-to-be-used registration screen SG includes a list display section 21 in which respective devices usable in the endoscope system are displayed in a list format and a selecting section 22 for selecting a device. A plurality of device names are displayed in the list display section 21. In FIG. 3, a scope (that is, the endoscope 2), the treatment instrument, the suction pump 6, the liquid feeding tank 7, and the UPD 5 are displayed. The selecting section 22 includes pulldown menu display sections arranged to correspond to the respective device names.

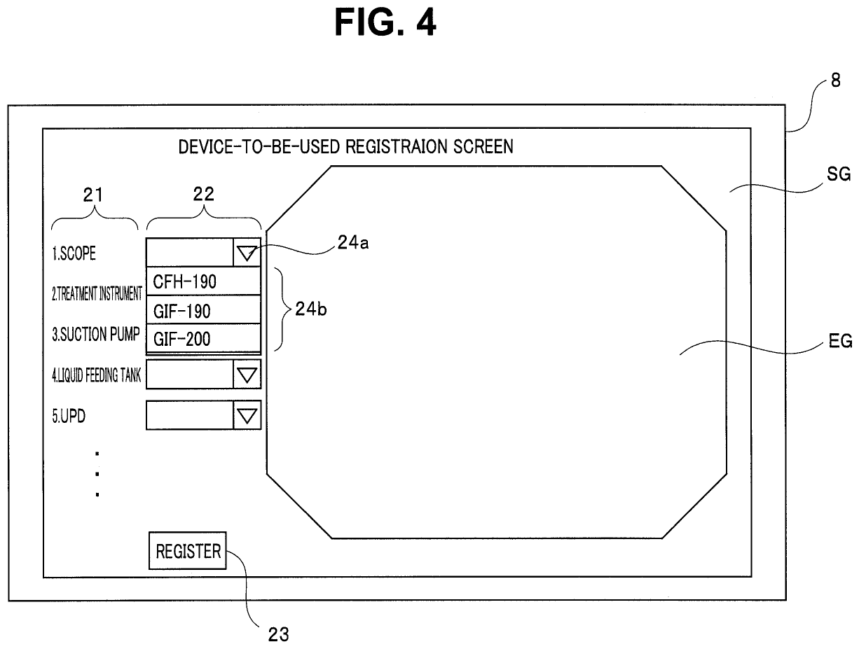

FIG. 4 is a diagram showing a display example of a pulldown menu display section at the time when a type of the endoscope 2 is set.

When the user moves a cursor to a pulldown display button 24a of the selecting section 22 corresponding to the scope and clicks the pulldown display button 24a, a pulldown menu display section 24b is displayed. The pulldown menu display section 24b displays a plurality of types of the endoscope 2 in a list format. The pulldown menu display section 24b also includes “not use”, that is, “OFF”.

When the user moves, using the mouse of the input apparatus 14, the cursor to a display region of a type of the endoscope 2 to be used out of the types displayed in the pulldown menu display section 24b and clicks the display region, the endoscope 2 of the type is selected. As a result, the endoscope 2 to be used by the user is set.

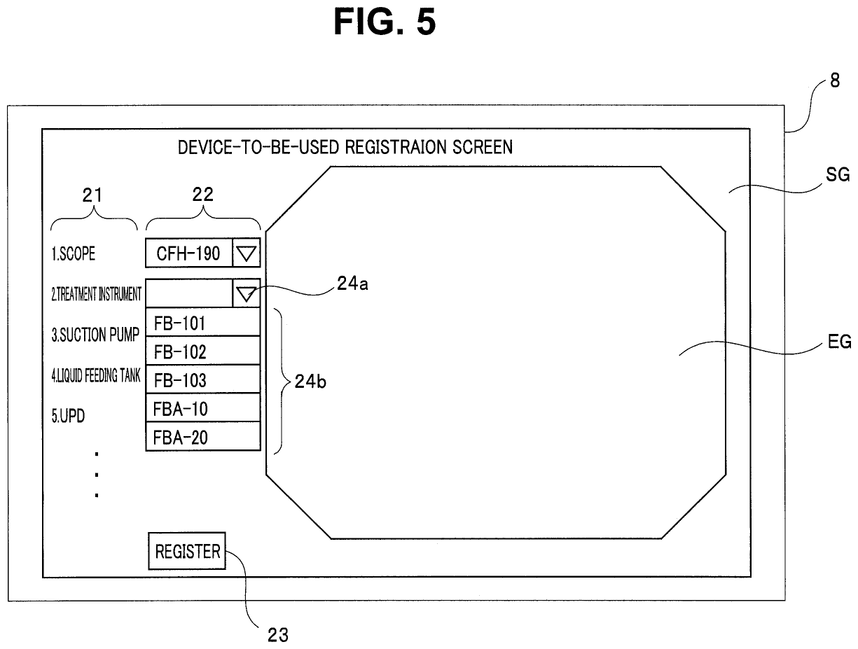

FIG. 5 is a diagram showing a display example of the pulldown menu display section at the time when a type of the treatment instrument, which is the accessory device, is set.

When the user moves the cursor to the pulldown display button 24a of the selecting section 22 corresponding to the treatment instrument and clicks the pulldown display button 24a, the pulldown menu display section 24b is displayed. The pulldown menu display section 24b displays a plurality of types of the treatment instrument in a list format. When the user moves, using the mouse of the input apparatus 14, the cursor to a display region of a type of the treatment instrument to be used out of the types displayed in the pulldown menu display section 24b and clicks the display region, the treatment instrument of the type is selected. As a result, the treatment instrument to be used by the user is set.

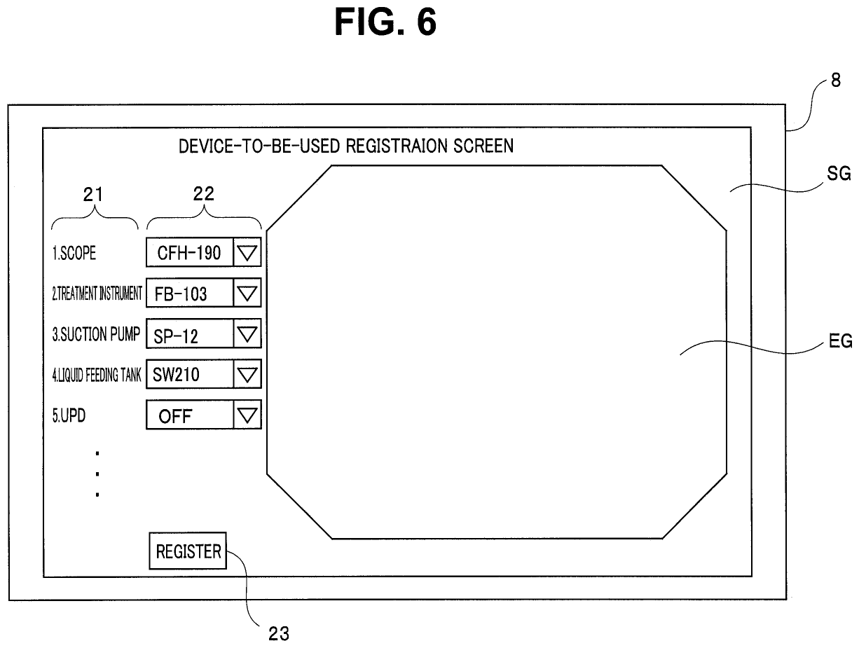

Similarly, the user can also set another treatment instrument, a suction pump, a liquid feeding tank, a monitor, and the like using the device-to-be-used registration screen SG. FIG. 6 is a diagram showing an example of a state in which devices to be used are selected on the device-to-be-used registration screen SG. In FIG. 6, types of devices selected by the user are displayed to correspond to device names. Note that in FIG. 6, “not use” (OFF) is selected for the UPD 5.

When finishing setting types and the like of all devices to be used, the user moves the cursor to the registration button 23 and clicks the registration button 23. Then, the devices selected using the pulldown menus are registered as devices to be used by the user in a test or the like to be carried out thereafter. With a scheme for selection from the pulldown menus, it is possible to prevent the user from erroneously registering a device that cannot be used at all.

As explained above, the user registers the types of the respective devices determined as the devices used in the test or the like.

Note that in the above explanation, the user selects the devices to be used out of the devices displayed in the pulldown menus. However, the user may directly input, from the keyboard or the like, a type of a device to be used.

### (Action)

Subsequently, an operation of the system configuration check of the endoscope system 1 explained above is explained.

### (Device-to-be-Used Registration Processing)

When the user inputs a device registration processing command in the keyboard of the input apparatus 14, the processor 11a reads out the device-to-be-used registration program UDR from the storage device 11b and executes the device-to-be-used registration program UDR.

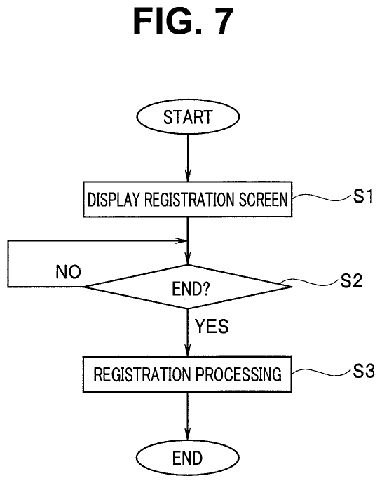

FIG. 7 is a flowchart showing an example of a flow of the device-to-be used registration processing by the device-to-be-used registration program UDR. When the device-to-be-used registration program UDR is executed, the processor 11a generates the device-to-be-used registration screen SG shown in FIG. 3 and displays the device-to-be-used registration screen SG on the monitor 8 (step (hereinafter abbreviated as S) 1). The user sets a device to be used as explained with reference to FIG. 4 and FIG. 5 using the device-to-be-used registration screen SG shown in FIG. 3.

The processor 11a determines whether the registration button 23 is clicked, that is, the registration ends (S2).

If the registration button 23 is not clicked (S2: NO), the processor 11a continuously displays the device-to-be-used registration screen SG without performing any processing.

If the registration button 23 is clicked (S2: YES), the processor 11a executes registration processing for registering, in the RAM or the storage device 11b, data of types and the like of all devices selected and set on the device-to-be-used registration screen SG (S3). As a result, data of names of respective accessory devices and types corresponding to the respective devices are recorded in the RAM or the like. Accordingly, the processing in S1 to S3 is performed for an information registering unit (the storage device 11b) of the processor 11a for registering data (second data) of devices to be used by the user.

### (System Configuration Check Processing)

After registering the devices to be used, for example, the user connects devices used for an endoscopic examination or the like to set the respective accessory devices in the test room. When the user inputs, from the keyboard of the input apparatus 14, a command for instructing a system configuration check for the devices to be used, the processor 11a reads out the system configuration check program SC from the storage device 11b and executes the system configuration check program SC.

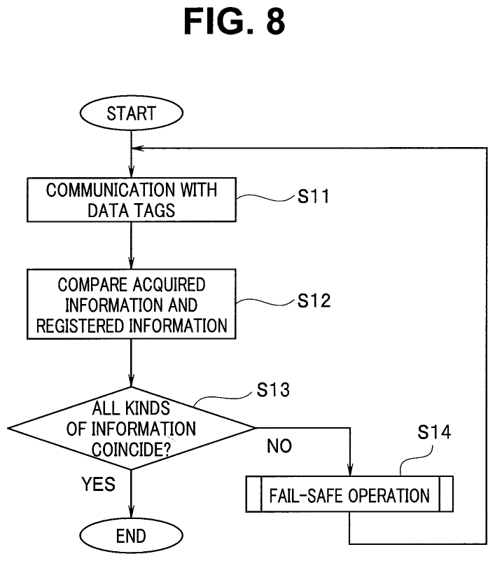

FIG. 8 is a flowchart showing an example of a flow of processing of the system configuration check program SC. The processor 11a performs communication by radio with respective data tags (S11). The processor 11a temporarily stores, in the RAM, data of the respective data tags acquired by performing communication with all the recognized data tags. For example, according to the communication scheme in which the passive microwave is used, data tags present in positions at approximately 2 meters or less from the processor 11a communicate with the processor 11a. Accordingly, the processing in S11 configures a reading section that is provided in the video processor 4 and reads the data of the respective data tags.

The processor 11a compares the data (first data) of all the data tags acquired in S11 and data (second data) of accessory devices registered in advance by the device-to-be-used registration processing (S12). The processor 11a determines whether the data (the first data) of all the data tags acquired in S11 and the data (the second data) of the devices set and registered in FIG. 5 coincide as a result of the comparison in S12 (S13). For example, if ten pieces of registered data (second data) are present, the processor 11a determines whether ten pieces of data (first data) are also acquired in S11 and the respective data coincide. More specifically, about the endoscope 2, the processor 11a determines whether a name and a type (second data) of the endoscope registered in the RAM are included in the received data (first data) from the respective data tags. Similarly, about the respective devices, the processor 11a determines whether names and types (second data) of the respective devices registered in the RAM are included in the received data (first data) from the respective data tags.

When the data of all the data tags acquired in S11 and the data of the registered devices coincide (S13: YES), the processor 11a does no processing. In other words, when the data (the second data) of the registered devices is included in the received data (first data) of all the data tags, the system configuration check program SC ends.

When the data (first data) of all the data tags acquired in S11 and the data (second data) of the devices registered in advance do not coincide (S13: NO), the processor 11a executes a predetermined fail-safe operation (S14). In other words, if not all of the registered devices are collected (S13: NO), a fail-safe operation (warning processing) is executed. In the fail-safe operation, warning by display on the monitor 8 that displays an endoscopic image is performed.

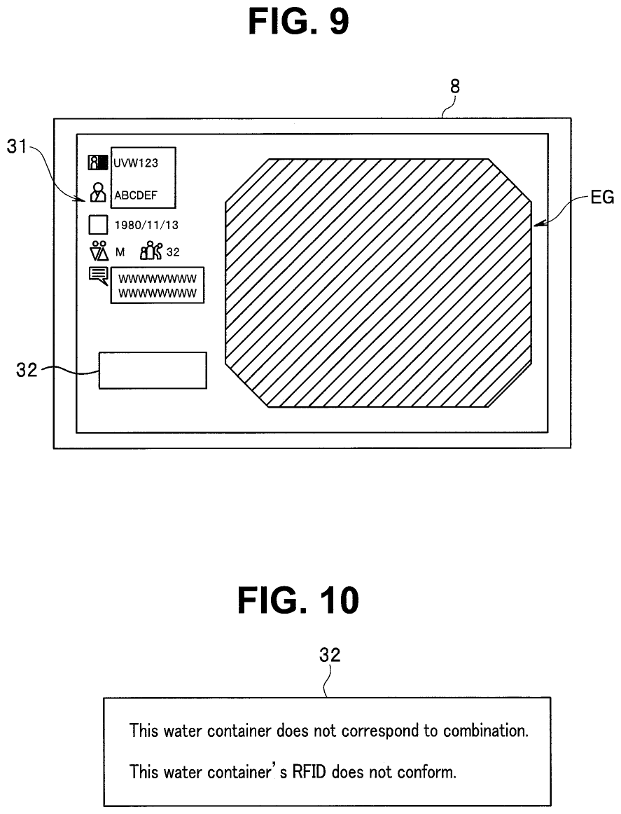

FIG. 9 shows a display example of the monitor 8, which is an example of the fail-safe operation. When the processor 11a is turned on, the device-to-be-used registration processing explained above can be executed. The processor 11a generates an image including, in a part of a screen, a various-kinds-of-information display section 31 that displays data such as a patient name and age and an endoscopic image display region EG and displays the image on the monitor 8. In FIG. 9, an endoscopic image is not displayed in the endoscopic image display region EG. The endoscopic image display region EG is in a pitch black state (blackout).

Thereafter, when the system configuration check processing is executed and the data (first data) of all the data tags acquired by the processor 11a and the data (second data) of the accessory devices registered in advance do not coincide, a message display section 32 for warning is displayed, for example, in red on the screen of the monitor 8 by the processing in S14. In other words, the warning is performed by displaying a predetermined message on the monitor 8.

FIG. 10 is a diagram showing a display example of a message in the message display section 32. As shown in FIG. 10, a message indicating that the liquid feeding tank is incorrect is displayed in the message display section 32. In other words, when the registered data (second data) and the acquired data (first data) do not coincide and data (third data) not included in the acquired data (first data) is present among the registered data (second data), the third data is displayed as warning. Therefore, the user can recognize that not all of the registered accessory devices are collected and a device not collected is, for example, the liquid feeding tank.

In particular, the endoscopic image display region EG is in the pitch black state and an endoscopic image is not displayed at all on the screen of the monitor 8. Accordingly, the user cannot perform an endoscopic examination. As a result, a test or the like in a state in which not all of the registered accessory devices are collected is not performed.

Note that the message display section 32 may be arranged on the endoscopic image display region EG. FIG. 11 is a diagram showing a display example of the monitor 8 that is an example of the fail-safe operation, in which the message display section 32 is arranged on the endoscopic image display region EG. In other words, the predetermined message in the message display section 32 is displayed in a display region for displaying an endoscopic image obtained by the endoscope 2 in the monitor 8, which is a display apparatus. The warning is performed by changing the display region, where the endoscopic image obtained by the endoscope 2 is displayed, on the monitor 8 to a predetermined display state (for example, blackout) without displaying the endoscopic image in the display region.

Furthermore, in the fail-safe operation, in addition to the message display on the monitor 8, beep sound or the like may be outputted together when the video processor 4 includes a buzzer or the like.

As explained above, the processing in S13 and S14 configures a warning section that, when the video processor 4 is turned on, compares the data (second data) respectively registered about the endoscope 2 and the accessory devices and the data (first data) of the plurality of data tags read in S11 and performs warning when the registered data (second data) and the read data (first data) do not coincide. In other words, the video processor, which is the control apparatus, includes the warning section.

After the fail-safe operation (S14), the processing returns to S11. When an appropriate device is set, after S11 and S12, when all the data coincide in S13 (S13: YES), that is, all of the registered accessory devices are collected, the fail-safe operation is not executed.

Note that when the data (third data) from the data tag of the accessory device not registered on the device-to-be-used registration screen SG is acquired, the data (third data) of the data tag not registered but acquired may be displayed on the monitor 8.

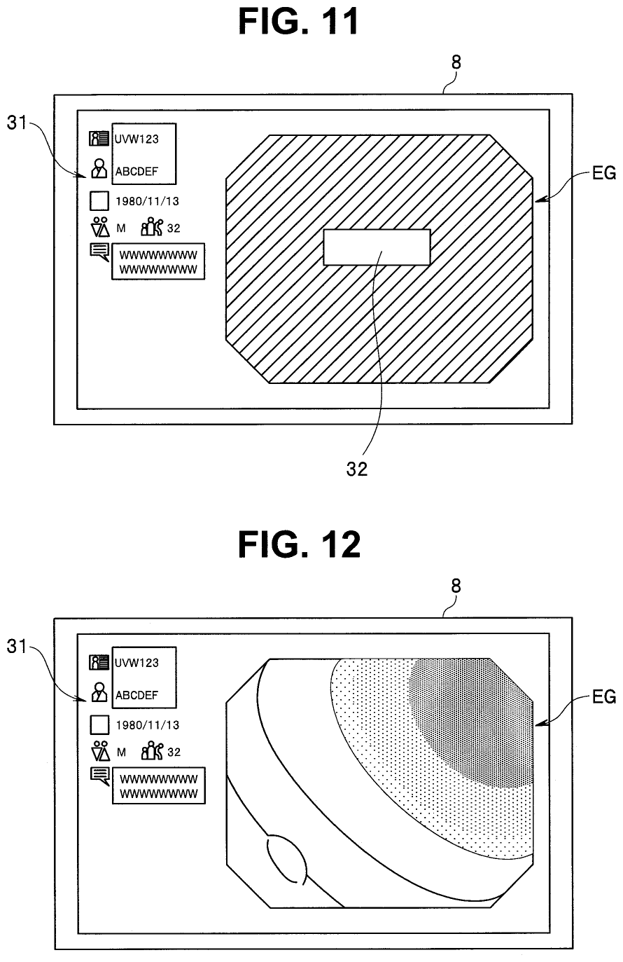

When all of the devices registered by the user are collected, the message display section 32 is not displayed and an endoscopic image is displayed on the endoscopic image display region EG. FIG. 12 is a diagram showing a display example of the monitor 8 at the time when all of the devices registered by the user are collected.

Therefore, if not all of the devices registered by the user are collected, a message is displayed and an endoscopic image is not correctly displayed on the monitor 8. Therefore, the user cannot start the test or the like.

Note that here, in the system configuration check, after the devices to be used are registered, when a predetermined button is pressed, the processor 11a communicates with all of the data tags and determines whether the respective devices coincide with the registered devices. However, after the devices to be used are registered, the processor 11a may always communicate with the respective data tags and perform a warning operation until all of the registered devices can be confirmed.

As explained above, according to the embodiment explained above, it is possible to provide an endoscope system, a control apparatus, an external apparatus, a combination of the control apparatus and the external apparatus, and a method of checking an endoscope system configuration that can easily confirm that not all of accessory device used in combination with an endoscope are correctly set.

In the embodiment explained above, if not all of the registered accessory devices are collected, since the warning operation is performed, the user does not perform the test or the like. In other words, at least one accessory device used in combination with the endoscope is different or lacks, it is possible to cause the user not to perform the test or the like.

When an accessory device not including a data tag is set, since an accessory device used in combination with the endoscope lacks, it is possible to cause the user not to perform the test or the like. This leads to preventing use of an accessory device, an operation of which is not guaranteed.

Note that in the embodiment explained above, the registration of the accessory devices is performed in the video processor 4 and the registered data of the accessory devices is stored in the storage device 11b of the video processor 4. However, data registration of the accessory devices may be performed by another apparatus (external apparatus). For example, registration processing may be able to be performed in an external apparatus such as a server apparatus connected to the video processor 4 via a network such as the Internet or a LAN. The registered data may be transmitted from the server apparatus to the video processor 4. In that case, for example, it is assumed that the registered data of the accessory devices is utilized as a determination material at the time of a diagnosis by a doctor in addition to an electronic medical record registered in a hospital. An information registering unit for registering the data (second data) of the devices to be used by the user may be a component of the external apparatus.

In the embodiment explained above, the processor 11a compares the data (first data) of all of the data tags acquired in S11 and the data (second data) of the devices registered by the device-to-be-used registration processing (S12). However, such comparison may be performed in the external apparatus 15 (see FIG. 2) such as a server apparatus connected to the video processor 4 via a network such as the Internet or a LAN. A comparison result may be transmitted from the external apparatus to the video processor 4. The video processor 4 can perform warning based on the comparison result received from the external apparatus. Consequently, it is possible to reduce a load and cost of the video processor 4 and provide a low-cost endoscope system.

Further, in the embodiment explained above, the accessory devices are only the accessory devices used in the endoscopic examination. However, the accessory devices may include devices (accessories) such as a brush used in cleaning and disinfection treatment for the endoscope 2 after the test or the like.

Even if the accessory devices used in combination with the endoscope conform to the endoscope, it is likely that a device not intended to use by the user is erroneously set.

For example, even if a set monitor conforms to the endoscope, that is, is usable, the user sometimes cannot perform an observation at assumed resolution. Even if set forceps conform to the endoscope, that is, are usable, if the forceps are not forceps intended by the user, a treatment instrument needs to be replaced because an assumed feeling of use, operation feeling, or the like is not obtained or assumed treatment cannot be performed.

The endoscope system 1 can easily confirm that not all of devices and the like that should be used in combination with the endoscope are correctly set.

The present invention is not limited to the embodiment explained above. Various changes, alterations, and the like are possible in a scope in which the gist of the present invention is not changed.

What is claimed is:

1. An endoscope system comprising:
an endoscope;
a control apparatus to which the endoscope is connected;
at least one accessory device used in combination with the endoscope and the control apparatus; and
data tags respectively provided in the endoscope and the accessory device, wherein
the control apparatus reads first data of the respective data tags, compares second data respectively registered about the endoscope and the accessory device and the first data, and performs warning when the first data and the second data do not coincide.

2. The endoscope system according to claim 1, wherein the warning is performed by display in a display apparatus that displays an endoscopic image obtained by the endoscope.

3. The endoscope system according to claim 2, wherein the warning is performed by displaying a predetermined message on the display apparatus.

4. The endoscope system according to claim 3, wherein the predetermined message is displayed in a display region, where the endoscopic image obtained by the endoscope is displayed, on the display apparatus.

5. The endoscope system according to claim 2, wherein the warning is performed by changing a display region, where the endoscopic image obtained by the endoscope is displayed, on the display apparatus to a predetermined display state without displaying the endoscopic image in the display region.

6. The endoscope system according to claim 1, wherein the control apparatus is a video processor to which the endoscope is connected.

7. The endoscope system according to claim 1, further comprising a plurality of accessory devices, wherein
when third data not included in the second data is present among the first data, the control apparatus displays the third data on a display apparatus that displays an endoscopic image.

8. The endoscope system according to claim 1, further comprising an information registering unit for registering the second data.

9. The endoscope system according to claim 8, wherein the information registering unit is provided in the control apparatus.

10. A control apparatus to which an endoscope is connected and that is used in combination with an accessory device,
the control apparatus reading first data of data tags respectively provided in the endoscope and the accessory device, comparing second data respectively registered about the endoscope and the accessory device and the first data, and performing warning when the first data and the second data do not coincide.

11. A control apparatus to which an endoscope is connected and that is used in combination with an accessory device and connected to an external apparatus via a network,
the control apparatus reading first data of data tags respectively provided in the endoscope and the accessory device and, based on a result of comparison by the external apparatus between second data respectively registered about the endoscope and the accessory device and the first data, performing warning when the first data and the second data do not coincide.

12. A combination of a control apparatus and an external apparatus to which an endoscope is connected and that are used in combination with an accessory device,
the control apparatus reading first data of data tags respectively provided in the endoscope and the accessory device,
the external apparatus comparing second data respectively registered about the endoscope and the accessory device and the first data, and
the control apparatus performing warning when the first data and the second data do not coincide.

13. The combination of the control apparatus and the external apparatus according to claim 12, wherein the second data is registered in the external apparatus.

14. An external apparatus to which a control apparatus to which an endoscope is connected and that is used in combination with an accessory device is connected via a network,
the external apparatus comparing first data of data tags respectively provided in the endoscope and the accessory device and second data respectively registered about the endoscope and the accessory device and transmitting a comparison result to the control apparatus.

15. A method of checking an endoscope system configuration, the endoscope system including an endoscope, a control apparatus to which the endoscope is connected, and an accessory device used in combination with the endoscope and the control apparatus,
the control apparatus reading first data of data tags respectively provided in the endoscope and the accessory device, comparing second data respectively registered about the endoscope and the accessory device and the first data, and performing warning when the first data and the second data do not coincide.

16. The method of checking the endoscope system configuration according to claim 15, wherein the second data is stored in the control apparatus.

17. The method of checking the endoscope system configuration according to claim 15, wherein the second data is stored in an external apparatus other than the endoscope system.

18. The method of checking the endoscope system configuration according to claim 17, wherein the external apparatus is a server apparatus connected to the control apparatus via a network.
